(19) United States
(12) Patent Application Publication
(10) Pub. No.: US 2009/0018587 A1
(43) Pub. Date: Jan. 15, 2009

(54) FRACTURE PLATE AND METHOD FOR FIXATION OF SAME TO A BONE SHAFT

(75) Inventor: Michael Bottlang, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: Apex Biomedical Company, LLC, Lake Oswego, OR (US)

(21) Appl. No.: 11/776,238

(22) Filed: Jul. 11, 2007

(51) Int. Cl. A61B 17/70; A61B 17/56; A61B 17/58

(52) U.S. Cl. 606/280; 606/286; 606/289

(57) ABSTRACT

Embodiments of the invention provide bone plates with an upper surface, a lower surface, and a longitudinal plate portion for fixation to a bone shaft. A longitudinal plate portion may have three or more non-collinear holes for locking screws, whereby the axis of at least one hole may be angled relative to the axis of another hole to improve the torsional strength of the plate-bone construct.

### TECHNICAL FIELD

Embodiments of the present invention relate to the field of orthopedics, more specifically, to an osteosynthesis plate and a method for bone fracture fixation by application of an osteosynthesis plate to a bone shaft.

### BACKGROUND

Various existing bone plates may be applied to bone with screws to span and stabilize a bone fracture. For fixation of bone fractures, the plate segment overlying the bone shaft typically has a collinear hole pattern oriented along the centerline of the plate. In some plates that utilize standard bone screws, non-threaded screw holes may be arranged in a staggered manner to increase the number of screw holes for a given plate length. However, threaded screw holes or screw holes adapted for locking screws are generally not utilized in a staggered alignment because such an alignment typically relies on variability of the approach angle for the screw to properly enter the bone, a function not provided by a typical locking screw and plate arrangement which has a substantially rigid direction of entry.

A locked plate is disclosed in French Patent 742,618 that has screw holes fitted with inside threads to enable locking screw fixation. By using locking screws with a corresponding threaded portion at the perimeter of the screw head, these locked plates enable positive locking at a fixed orientation between the threaded plate holes and the locking screws. For fixation on the shaft portion of long bones, these plates have a collinear hole pattern oriented along the centerline of the plate to ensure that fixed locking screws penetrate through the midline of the bone shaft. However, since the locking screws are inserted in the bone shaft in a collinear and parallel manner, torsional forces around the single plane of fixation may cause screw bending and/or further damage to the bone.

In some large plates for fixation of fractures of the femur and humerus, locking screw holes are moderately non-collinear having a slight offset from the plate midline. However, this slight offset is typically less than half of the diameter of the screw hole and still utilizes a straight approach of the screws (i.e., all screws are parallel to each other).

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIG. 1 illustrates three perspective views of a representative plate and screw construct for fixation of bone in accordance with various embodiments of the present invention;

FIGS. 2, 3, 4 and 5 illustrate cross-sectional views of various embodiments of the present invention in association with a bone shaft;

FIG. 6 illustrates top views of various exemplary hole patterns of a plate member in accordance with embodiments of the present invention;

FIG. 7 illustrates fixation of a bone fracture with a plate and screw construct in accordance with various embodiments of the present invention; and

FIG. 8 illustrates a cross-sectional view of a plate and screw construct in accordance with various embodiments of the present invention.

### DETAILED DESCRIPTION OF EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the present invention, the phrase “A/B” means A or B. For the purposes of the present invention, the phrase “A and/or B” means “(A), (B), or (A and B)”. For the purposes of the present invention, the phrase “at least one of A, B, and C” means “(A), (B), (C), (A and B), (A and C), (B and C), or (A, B and C)”. For the purposes of the present invention, the phrase “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

For the purposes of describing embodiments of the present invention, the term “osteosynthesis” refers to a device or procedure that stabilizes and/or joins the ends of fractured bones, in part, using mechanical devices such as plates, pins, rods, splints, wires or screws.

For the purposes of describing embodiments of the present invention, the terms “fixation” or “fixing” refer to the stabilization of some or all of the parts of a fractured bone.

For the purposes of describing embodiments of the present invention, the term “locking screw” means a screw that has a mechanism for rigid or substantially rigid engagement with a corresponding screw hole in an osteosynthesis plate, thereby residing at a predetermined angle with respect to the osteosynthesis plate. The term “predetermined angle” is used above to describe a locking screw's angle of residence in the plate, however, in embodiments, a locking screw may be provided with a mechanism, such as a reduced shaft size, to allow for a small degree of lateral movement in the screw shaft. Thus, a predetermined angle may change slightly due to flexibility of the shaft, space in the near cortex through-hole, etc. while still being considered predetermined.

In order to improve the strength of standard locked plating constructs under torsional loading, embodiments of the present invention describe locked plates that generate a multi-planar fixation by provision of a non-collinear pattern of screw holes along the plate portion overlying the bone shaft. In embodiments, to ensure that locking screws will penetrate approximately through the midline of the bone shaft or directed toward such an alignment, plate hole axes may be aligned to provide convergence of one or more screws with the midline of the bone shaft, or more generally directed toward a plane aligned with the longitudinal axis of the plate.

Non-collinear and diverging/converging orientations of locking screws are used for uni-cortical fixation of plates to spinal vertebrae (e.g., U.S. Pat. No. 6,595,993) or for metaphyseal fixation at the ends of long bone in which screws penetrate only the bone surface underlying the plate but not the opposite bone surface. Embodiments of the present invention, however, are concerned with plate fixation to the shaft portion of long bones (diaphysis), wherein the fixation screws penetrate the bone surface underlying the plate as well as at least partially into the opposite bone surface. In embodiments, such screws may be referred to as bi-cortical fixation screws. For plate fixation to the shaft portion of long bones, locating screw holes for bi-cortical fixation screws in a non-collinear arrangement, instead of only along the longitudinal centerline of the plate, provides beneficial torsion resistance and excellent strength/support.

In embodiments, far-cortical locking screws may be used. Any suitable far-cortical locking screws may be used in embodiments of the present invention, such as those described in U.S. patent application Ser. No. 11/058,935, the entire contents of which are hereby incorporated by reference.

Embodiments of the present invention provide an osteosynthesis plate and a method for fixing a bone fracture using an osteosynthesis plate, whereby a portion of the osteosynthesis plate or all of the osteosynthesis plate extends along the shaft of a long bone. In embodiments of the present invention, an osteosynthesis plate may be placed across the bone fracture and fixed with bone screws to the bone on each side of the fracture to provide bone alignment and stability.

Thus, in an embodiment, there is provided an osteosynthesis plate, comprising a longitudinal plate having an upper surface and a lower surface, wherein the lower surface has a bone-surface contour, wherein the plate has at least three non-collinear holes, each hole providing a passage and engagement for an associated bone screw and providing a predetermined angle of orientation of the associated bone screw, at least two of the at least three non-collinear holes having different predetermined angles of orientation.

For the purposes of describing embodiments of the invention, the term “bone-surface contour” refers to a shape of the lower surface of an osteosynthesis plate that is similar to or matches the underlying bone (i.e., the bone(s) on which the plate is intended to be used), or is designed to aid in aligning the plate and/or screws with the bone. In an embodiment, a bone-surface contour may be flat or may provide a transverse curvature (curved around the longitudinal axis of the plate) to provide better alignment of the screw holes with the bone. In embodiments, a bone-surface contour may be uniform along the plate or may differ in regions. Despite the presence of a bone-surface contour in the lower surface of a plate, the plate, when in use, may contact or may be suspended above the bone, or the contact parameters may differ along the length of the plate.

In embodiments of the present invention, bone screws may be used that have a mechanism for rigid, locking engagement with holes in the osteosynthesis plate. In an embodiment, the holes of the osteosynthesis plate may have threads that correspond to the screw threads of the bone screws. In embodiments, the threads of the screw holes and the threads of the screws may be timed such that a particular desired orientation/alignment of the screw may be achieved when the screw is inserted completely. In an embodiment, using complementary timed threads are particularly beneficial when using screws that have rotationally asymmetric shafts providing different flexibility characteristics in different directions, such as further described in U.S. patent application Ser. No. 11/672,300, filed Feb. 7, 2007, the entire contents of which are hereby incorporated by reference. In an embodiment, locking screws may be used on only one or both sides of a fracture site.

For the purposes of describing embodiments of the present invention, the term “rotationally asymmetric shaft” refers to a shaft of a screw that has a different flexibility or bending characteristic depending on the direction of a force applied. This may be accomplished for example with a non-circular cross-section.

Alternatively, in embodiments, a screw having a rotationally symmetric cross-sectional shaft may be used, whether or not the screw is a locking screw, and whether or not a screw is unicortical, bicortical, or far cortical locking. In an embodiment using a far cortical locking screw having a rotationally symmetric cross-section, the near cortex may be predrilled, or caused to open larger due to integrated features (self-tapping features or flutes) on the screw, to provide a hole in the near cortex that is larger than the diameter of the screw shaft residing in the opening of the near cortex. In such an embodiment, the screw retains a degree of flexibility since the screw is not locked into the near cortex and a small amount of room is provided around the shaft within the near cortex hole. In embodiments, a rotationally symmetric screw may have a uniform cross-section along the shaft, or there may be portions of the shaft with larger cross-sections and portions with smaller cross-sections. For example, in an embodiment, the leading end of the screw may have a larger cross-section and the portion of the screw that will reside in the near cortex when in use may have a reduced cross-sectional diameter.

In an embodiment, at least some of the screw holes in an osteosynthesis plate may be positioned in a non-collinear manner. In other words, in an embodiment, three or more screw holes may be provided in an osteosynthesis plate in which at least one of the holes does not reside on the same line as a line on which two other holes reside. Thus, in an embodiment, the screw holes of an osteosynthesis plate may be provided in a non-collinear (staggered) arrangement. In embodiments, this type of arrangement provides for multi-planar screw fixation in the bone and greatly enhances the construct strength as compared to collinear screw placement since bending or torsional loading may cause bending of collinear screws.

Additionally, in an embodiment, a non-collinear arrangement of screws and screw holes provides a non-collinear hole pattern in the bone surface underlying the plate which may reduce the risk for fracture propagation between the screw holes.

In embodiments, a non-collinear arrangement of screws and screw holes includes situations in which some holes are collinear and also encompasses various degrees of staggering, whether regular or irregular.

In embodiments of the present invention, bone screws may be configured in a converging manner to penetrate the bone shaft in proximity to or toward the midline of the bone shaft (the midline in this description being in the transverse direction of the bone as opposed to longitudinal). Such embodiments may be accomplished by angling the entry and alignment of the screws toward the midline of the bone shaft. In an embodiment, an osteosynthesis plate may have a curved transverse cross-section to provide alignment of the screw holes with the transverse midline of the bone shaft or more generally directed inward toward a plane aligned with the longitudinal axis of the plate.

In conjunction with the converging orientation of the screws, in an embodiment, bi-cortical locking screws may be utilized thus providing a strong screw fixation in the bone surface underlying the plate and in the opposing bone surface. In other embodiments, far-cortical locking screws may be used in conjunction with a converging orientation of screws.

For the purposes of describing embodiments of the present invention, the term “converging” refers to a direction of a bone screw that is associated with a screw hole located off the longitudinal midline of an osteosythesis plate and has an angle of insertion that is generally toward, and, in embodiments, across the longitudinal midplane of the osteosynthesis plate. The concept of converging bone screws may be used to describe multiple screws that are inserted into an osteosynthesis plate at different partially opposing angles such that the insertion directions of the various screws converge. In embodiments, two or more bone screws may converge on a plane that is aligned with the longitudinal axis of the osteosynthesis plate and the bone shaft. In such an embodiment, such screws may converge on the plane within the bone shaft, or, in other embodiments, may converge on the plane on a point that is extrapolated outside the bone shaft.

In embodiments, converging screws need not all be uniformly converging, i.e. have the same angle of entry. Thus, in embodiments, two or more bone screws may converge on a plane that is not aligned with the longitudinal axis of the osteosynthesis plate and the bone shaft.

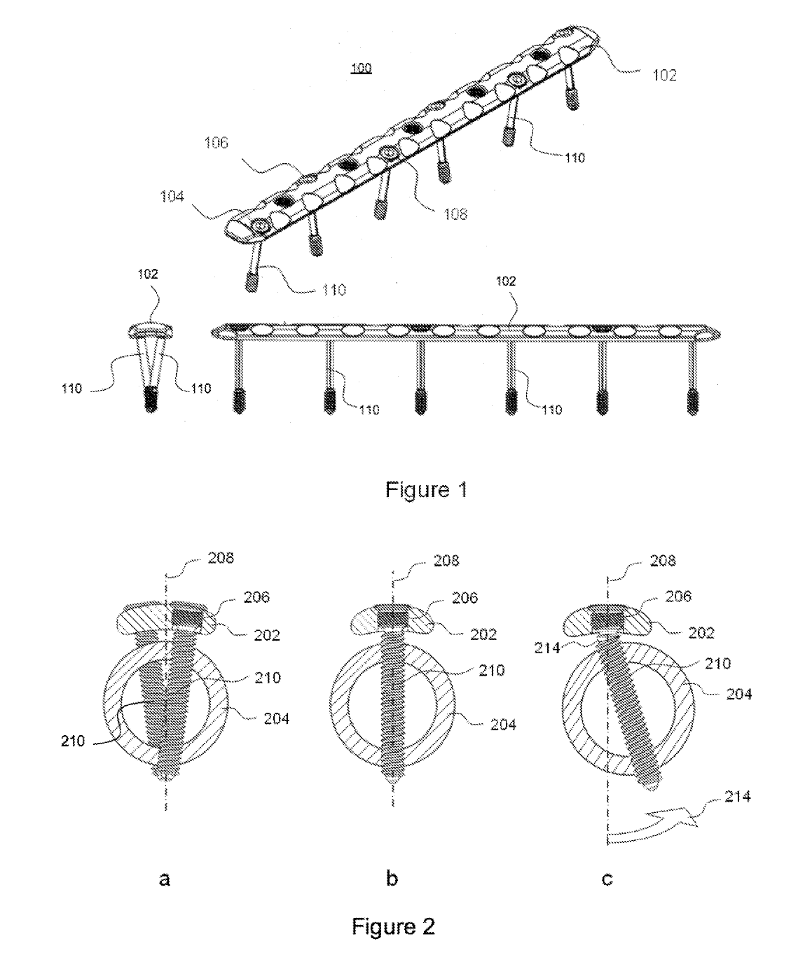

FIG. 1 shows an exemplary apparatus 100 for fixing a fractured bone with an osteosynthesis plate 102. Osteosynthesis plate 102 may have three or more non-collinear screw holes 104, 106, and 108 with a mechanism for rigid, locking engagement with bone screws 110. For this purpose, in an embodiment, screw holes 104, 106, and 108 may be fitted with an inside thread to engage with a corresponding thread on the head segment of bone screws 110.

As shown in FIG. 1, osteosynthesis plate 102 may be provided with a curvature (an example of a bone-surface contour) along the length of plate 102 (around the longitudinal axis of the plate) to better fit to the diaphysis and/or to provide better alignment with the bone.

FIG. 2a shows a cross-sectional view of an exemplary system for affixing an osteosynthesis plate 202 to bone shaft 204. Screw hole 206 and other screw holes are placed in a non-collinear manner at a distance from the longitudinal midline 208 of osteosynthesis plate 202. Screws 210 may engage osteosynthesis plate 202 in an orientation that enables convergence of the screw shafts. In the presence of three or more non-collinear screws, this multi-planar screw fixation may increase the strength of the fixation construct. In the absence of non-collinear screw placement, screw 210 may enter bone shaft 204 in a single plane, as shown in FIG. 2b. As a consequence, bending and torsional forces 214 to the single-plane fixation construct may induce bending of the shaft of screw 210 as depicted in FIG. 2c which may cause further damage to the bone and/or hinder the healing process, and/or may cause healing in a mal-aligned orientation.

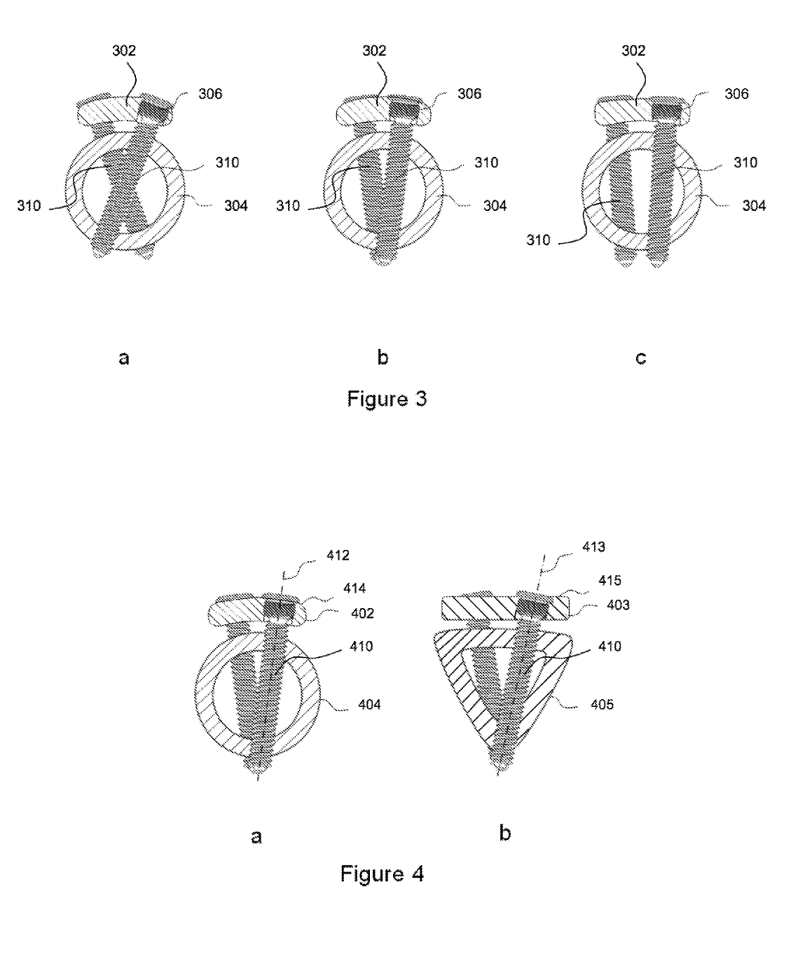

FIG. 3 shows an exemplary system in accordance with an embodiment of the present invention showing an osteosynthesis plate 302 in association with a cross-section of bone shaft 304. In embodiments of the present invention, plate hole 306 and other plate holes for locking screws 310 may be designed with various angles with respect to the longitudinal midline of osteosynthesis plate 302 resulting in various degrees of convergence of locking screws 310 in bone shaft 304. In an embodiment, locking screws 310 converge to a degree whereby the projected planes of the locking screws in the longitudinal direction of osteosynthesis plate 302 intersect within bone shaft 304 (FIG. 3a). In another embodiment, locking screws converge to a degree whereby the planes of the locking screws intersect an imaginary longitudinal line at or near the bone surface (far cortex) opposite to the plate (FIG. 3b). In another embodiment, locking screws converge to a degree whereby the planes of the locking screws do not intersect within the cross-section of the bone shaft (FIG. 3c), but rather would intersect if extrapolated beyond bone shaft 304.

In embodiments, the angle for any one screw, as well as the resulting degree of convergence of multiple screws, may be controlled by the curvature of the osteosythesis plate and/or the orientation of the screw holes in the osteosynthesis plate. In embodiments, the curvature of an osteosynthesis plate may be regular or irregular in the transverse and/or longitudinal direction, for example, to account for anatomical differences in bones.

Embodiments of the present invention are applicable to any of a variety of long bones found in a human or animal body, whether straight or curved, and having various cross-sections. In embodiments, osteosynthesis plates are configured for use on the diaphysis, although, in embodiments, such an osteosynthesis plate may also have a portion that extends into or along the metaphysis, which is typically referred to as a periarticular plate.

FIG. 4 shows an exemplary system in accordance with an embodiment of the present invention with an osteosynthesis plate 402 or 403 in association with bone shaft 404 or 405 with various cross-sectional geometries (FIGS. 4a and 4b, respectively). In an embodiment of the present invention as shown in FIG. 4a, osteosynthesis plate 402 may have a substantially curved cross-sectional geometry (transverse curvature) approximating the transverse curvature of underlying bone shaft 404, whereby axis 412 of associated locking screw 410 is located substantially perpendicular to surface 414 of osteosynthesis plate 402 (at the location of the screw hole). In another embodiment of the present invention as shown in FIG. 4b, osteosynthesis plate 403 may have a substantially flat cross-sectional geometry approximating the surface of underlying bone shaft 405, whereby axis 413 of associated locking screw 410 is angled in a converging manner and angled relative to surface 415 of osteosynthesis plate 403. In FIG. 4b, surface 415 may have a recess configured to accept locking screw 410 in a fully or partially recessed manner. Such an embodiment permits locking screw 410 to be aligned at an angle with respect to surface 415 without having any of or a substantial portion of the head of screw 410 extend above surface 415.

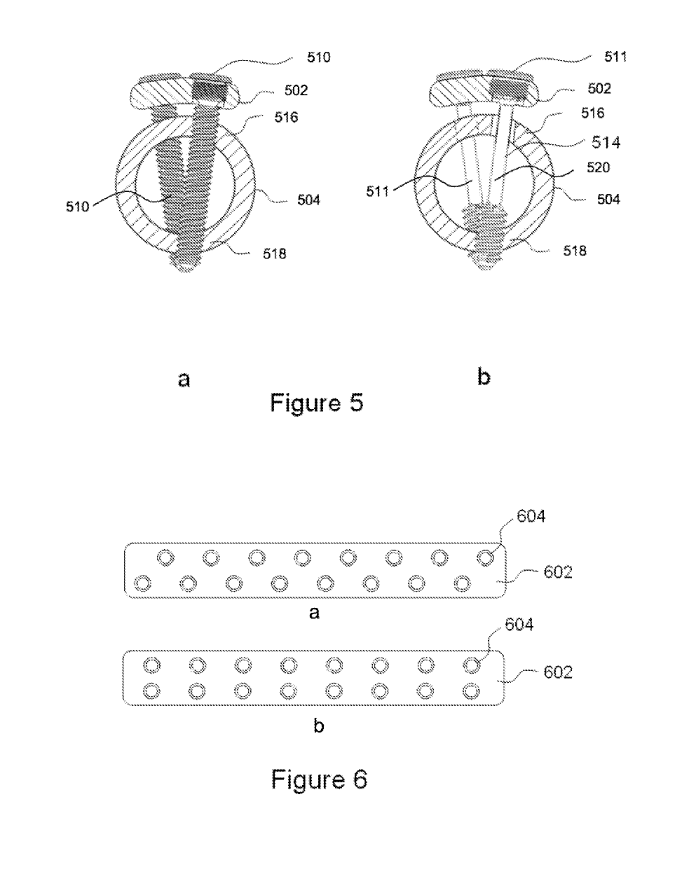

FIG. 5 shows alternative embodiments of the present invention with a cross-sectional view of an osteosynthesis plate 502 in association with a bone shaft 504. In an embodiment, osteosynthesis plate 502 is coupled to bone shaft 504 using locking screws 510, whereby screws 510 engage in near cortex 516 underlying plate 502 and in the opposing or far cortex 518 of bone shaft 504 (FIG. 5a). In another embodiment, osteosynthesis plate 502 is coupled to bone shaft 504 using far cortical locking screws 511 (FIG. 5b). Far cortical locking screws 511 are partially threaded and lock into plate 502 and into far cortex 518 of bone shaft 504 but not in near cortex 516 underlying plate 502. Due to the reduced shaft diameter of far cortical locking screws 511 at the non-threaded section 520, the screw shaft of far cortical locking screws 511 may undergo a small degree of flexion before contacting the sides of the pass-through holes in near cortex 516 thus providing added support and construct stiffness, while maintaining a small degree of flexion. This controlled, small degree of flexion allows plate 502 and screws 511 to absorb some torsion while promoting bone healing.

As indicated above, the small degree of flexion provided with far cortical locking screws 511 may also be provided by over-drilling the near cortex (making a hole in the near cortex larger than the portion of the shaft of the screw residing in the near cortex) and using a far cortical locking screw with or without a reduced diameter shaft.

FIG. 6 shows alternate patterns of holes 604 in osteosynthesis plate 602 in accordance with an embodiment of the present invention. In an embodiment of the present invention, holes 604 may be staggered (FIG. 6a). In another embodiment, holes may be located in a side-by-side, aligned pattern (FIG. 6b). In an embodiment, holes may be located in a non-uniform pattern throughout a plate. In embodiments, one or more additional holes may be present along the longitudinal midline of the plate. In embodiments, one or more screw holes may have means for locking screw engagement. In embodiments, the hole patterns may extend over a portion of the osteosynthesis plate or over the entire length of the osteosynthesis plate.

In embodiments as described above, one or more screw holes may be offset from the longitudinal midline of the plate. In an embodiment, to increase the torsion resistance, the extent of the offset may be large, and, in embodiments, may be maximized or optimized. In an embodiment, a screw hole may be offset from the longitudinal midline of the plate such that no portion of the screw hole overlaps the midline of the plate. In an embodiment, a screw hole may be offset from the midline by a distance greater than the diameter of the screw hole, for example approximately 1, 2, or 3 times the diameter of the screw hole.

In addition, in an embodiment, while osteosynthesis plate 602 is shown with a substantially uniform shape, different shapes may be utilized. For example, in an embodiment, one or both ends of osteosynthesis plate 602 may flare to accommodate the metaphysis of a bone. In embodiments, a flared portion of osteosynthesis plate 602 may have screw holes for directional, angled entry, whether regular, irregular, converging, and/or diverging, etc. in the angles and/or direction of entry.

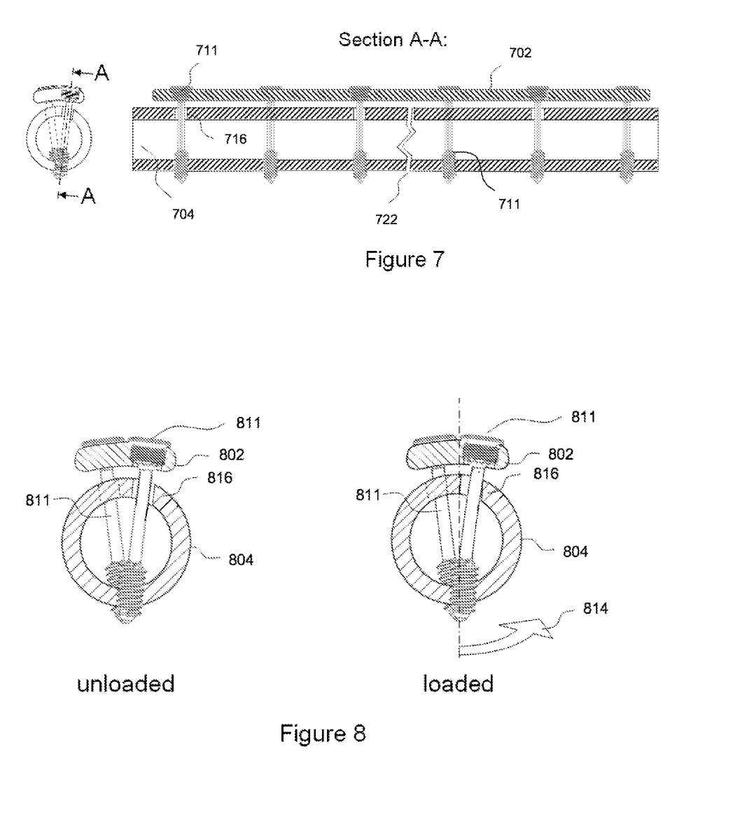

FIG. 7a shows an embodiment of the present invention with osteosynthesis plate 702 in association with a bone shaft 704. In this embodiment, osteosynthesis plate 702 is attached with far cortical locking screws 711 to one or both sides of the fracture location 722 in bone shaft 704. Since far cortical locking screws 711 do not rigidly engage in near cortex 716 underlying the plate, the shafts of screws 711 may undergo a small degree of flexion before contacting near cortex 716 for added support. This motion will provide a small, defined envelope of elastic flexibility. Such flexible fixation may provide substantial advantages over the more rigid fixation with bi-cortical locking screws. For example, the flexible construct may improve the load distribution and thereby prevent stress concentrations and improve construct strength. Furthermore, flexible constructs allow for small amounts of bony movement at the fracture site which is known to stimulate and enhance the healing process. Conversely, too rigid fixation with locking screws may suppress bony movement and fracture healing.

FIG. 8 depicts a cross-sectional view of an embodiment with far cortical locking screws 811. Under torsional and bending loads 814, the non-collinear screws enable a small amount of displacement of bone shaft 804 relative to plate 802 until the shafts of screws 811 contact the near cortex 816 for added support. This embodiment may provide a desired flexible construct for situations in which far cortical locking screws 811 are used on either one side of the bone fracture or on both sides of the bone fracture.

In embodiments, methods of fixing bones using an osteosynthesis plate and screw construct are also provided. In an embodiment, a method is provided comprising placing an osteosynthesis plate on a fractured bone across a fracture site, wherein the osteosynthesis plate has a longitudinal plate having an upper surface and a lower surface, wherein the lower surface has a bone surface contour and wherein the plate has at least three non-collinear holes, each hole providing a passage and engagement for an associated bone screw and providing a predetermined angle of orientation of the associated bone screw, at least two of the at least three non-collinear holes having different predetermined angles of orientation, and inserting locking screws into each of the at least three non-collinear holes and into the bone.

In embodiments of the present invention, osteosynthesis plates may have any suitable length. In an embodiment, the length of an osteosynthesis plate may be sufficient for fixation with two or more screws on each side of the fracture. Exemplary lengths of an osteosynthesis plate may be about 20-300 mm, for example, about 50-150 mm.

Osteosynthesis plates in accordance with embodiments of the present invention may have any suitable number of screw holes. In embodiments, the screw holes may have a regular pattern or all or some of the pattern may be irregular.

Osteosynthesis plates according to embodiments of the present invention may have any suitable width. In an embodiment of the present invention, the width of an osteosynthesis plate may be substantially constant along its length, or may vary at one or more locations, for example, to facilitate easier insertion, or to alter the bendability or flexibility in particular regions. For example, a plate segment may have a width of about 5-30 mm, for example 10-20 mm, among others.

An osteosynthesis plate in accordance with embodiments of the present invention may have any suitable thickness. In an embodiment of the present invention, the thickness may be substantially constant along the length of an osteosynthesis splint, or may vary at one or more locations, for example, to facilitate easier insertion, or to alter the bendability or flexibility in particular regions. Exemplary thicknesses for osteosynthesis plates may be about 1-10 mm, for example, about 3-7 mm.

In an embodiment of the present invention, an osteosynthesis plate may have any suitable in-plane or out-of-plane curvature, such as a transverse curvature and/or a longitudinal curvature, or may lack a curvature. In an embodiment of the present invention, a curvature of the entire osteosynthesis plate or segments thereof may be similar to, or matched to, the curvature of a portion of a bone shaft.

In embodiments of the present invention, osteosynthesis plates and screws may be made, for example, of a malleable and bioinert material, such as medical grade titanium (Ti6Al4V) or stainless steel (316L). In an embodiment of the present invention, an osteosynthesis plate may be constructed of a material being sufficiently malleable to allow for perioperative adjustment to conform the plate to a particular bone geometry.

In embodiments of the present invention, osteosynthesis plates may be permanent or removable.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. An osteosynthesis plate, comprising:
a longitudinal plate having an upper surface and a lower surface, wherein said lower surface has a bone-surface contour,
wherein said plate has at least three non-collinear holes, each hole providing a passage and engagement for an associated bone screw and providing a predetermined angle of orientation of the associated bone screw, at least two of said at least three non-collinear holes having different predetermined angles of orientation.

2. The plate of claim 1, wherein the bone-surface contour of the lower surface of the plate has a curved transverse cross-section along at least a portion of the plate.

3. The plate of claim 1, wherein the longitudinal plate has a shape configured to fit to a longitudinal curvature of a bone shaft.

4. The plate of claim 1, wherein at least one of the non-collinear holes has a predetermined angle of orientation directed toward a longitudinal midplane of the plate.

5. The plate of claim 1, wherein a plurality of the non-collinear holes are arranged in a staggered pattern.

6. The plate of claim 5, wherein at least one of the holes is offset from a longitudinal midline of the plate.

7. The plate of claim 6, wherein said at least one of the holes offset from a longitudinal midline of the plate is offset from the longitudinal midline of the plate such that the hole does not overlap with the longitudinal midline of the plate.

8. The plate of claim 5, wherein the pattern extends over only a portion of the plate.

9. The plate of claim 1, wherein a plurality of the non-collinear holes are arranged in a side-by-side pattern.

10. The plate of claim 9, wherein at least one of the holes is offset from a longitudinal midline of the plate.

11. The plate of claim 10, wherein said at least one of the holes offset from a longitudinal midline of the plate is offset from the longitudinal midline of the plate such that the hole does not overlap with the longitudinal midline of the plate.

12. The plate of claim 9, wherein the pattern extends over only a portion of the plate.

13. The plate of claim 1, wherein said engagement for an associated bone screw comprises threads in the holes for engagement with threads on the associated bone screw.

14. The plate of claim 11, wherein said threads in the holes comprise threads for engagement with locking screws.

15. The plate of claim 12, wherein the threads in the holes are timed with the threads of the locking screws to provide a predetermined rotational alignment of the locking screws when engaged fully with the holes.

16. An osteosynthesis plate and screw construct, comprising:
a longitudinal plate having an upper surface and a lower surface, wherein said lower surface has a bone-surface contour,
wherein said plate has at least three non-collinear holes, each hole providing a passage and engagement for an associated bone screw and providing a predetermined angle of orientation of the associated bone screw, at least two of said at least three non-collinear holes having different predetermined angles of orientation; and
at least three locking screws for coupling the plate to a bone.

17. The construct of claim 16, wherein said locking screws are adapted to engage in a near and far cortex of an associated bone.

18. The construct of claim 16, wherein said locking screws are adapted to engage only in a far cortex of a bone and to retain flexibility of movement relative to a near cortex of a bone.

19. The construct of claim 18, wherein at least one of said locking screws has a shaft with a rotationally symmetric cross-section.

20. The construct of claim 18, wherein at least one of said locking screws has a shaft with a rotationally asymmetric cross-section along at least a portion of the shaft.

21. The construct of claim 18, wherein at least one of said locking screws has a shaft with a portion residing in the near cortex, said portion of the shaft having a diameter less than a diameter of at least one other portion of the shaft.

22. A method for fixation of an osteosynthesis plate to a bone, comprising:
placing an osteosynthesis plate on a fractured bone across a fracture site, wherein the osteosynthesis plate has a longitudinal plate having an upper surface and a lower surface, wherein said lower surface has a bone-surface contour and wherein said plate has at least three non-collinear holes, each hole providing a passage and engagement for an associated bone screw and providing a predetermined angle of orientation of the associated bone screw, at least two of said at least three non-collinear holes having different predetermined angles of orientation; and
inserting locking screws into each of said at least three non-collinear holes and into the bone.

23. The method of claim 22, wherein said locking screws engage in a near and far cortex of the bone.

24. The method of claim 22, wherein said locking screws engage only in a far cortex of the bone and retain flexibility of movement relative to a near cortex of the bone.

25. The method of claim 24, wherein at least one of said locking screws has a shaft with a rotationally symmetric cross-section.

26. The method of claim 24, wherein at least one of said locking screws has a shaft with a rotationally asymmetric cross-section along at least a portion of the shaft.

27. The method of claim 24, wherein at least one of said locking screws has a shaft with a portion residing in the near cortex, said portion of the shaft having a diameter less than a diameter of at least one other portion of the shaft.

28. The method of claim 22, wherein at least one of said locking screws has a predetermined angle of orientation directed toward a longitudinal midline of the bone.

29. The method of claim 22, further comprising, prior to inserting locking screws into the bone, predrilling at least one hole in a near cortex of the bone to accommodate a locking screw.

30. The method of claim 29, wherein said locking screw has at least one integrated feature to expand the predrilled hole in the near cortex when said locking screw is inserted into the predrilled hole.

31. The method of claim 29, wherein said predrilled hole has a diameter larger than a diameter of a shaft of the locking screw.
